(19) United States
(12) Patent Application Publication
(10) Pub. No.: US 2003/0083557 A1
(43) Pub. Date: May 1, 2003

(54) Method and system for remote monitoring of the effect of a medicament

(76) Inventor: Volker Schmidt, Erlangen (DE)

Correspondence Address: YOUNG & THOMPSON, 745 SOUTH 23RD STREET 2ND FLOOR, ARLINGTON, VA, 22202

(21) Appl. No.: 10/263,690

(22) Filed: Oct. 4, 2002

(30) Foreign Application Priority Data
Oct. 4, 2001 (DE) 101 48 838.6

(51) Int. Cl. A61B 5/00

(52) U.S. Cl. 600/300

(57) ABSTRACT

A method and system for remote monitoring of the effect of a medicament taken by a person. The system includes a data bank for storing an electronic monitoring file allocated to the person and to the medicament and for storing data suitable for a description of an expected effect of the medicament, and elements for receiving data suitable for the description of the actual effect of the medicament and elements for comparing the data suitable for the description of the actual effect of the medicament with the data suitable for the description of the expected effect of the medicament. A computer program which implements the method and the system according and a data processing unit on which the computer program is stored are disclosed.

### FIELD OF THE INVENTION

The invention relates to a method and a system for remote monitoring of the effect of a medicament which a person is taking.

### BACKGROUND OF THE INVENTION

A physician generally prescribes a patient a medicament so that the patient's state of health improves or at least does not deteriorate. The physician can of course check the effect of the medicament if the patient consults him while taking the medicament or after taking the medicament. However, systematic monitoring of the effect of the prescribed medicament is often not possible.

### SUMMARY OF THE INVENTION

It is therefore an object of the invention to make available a method which permits straightforward and systematic monitoring of the effect of a medicament. A further object of the invention is to make available a system which permits straightforward and systematic monitoring of the effect of the medicament.

The first object of the invention is achieved by a method for remote monitoring of the effect of a medicament, said method comprising the following method steps:

a) setting up an electronic monitoring file which is allocated to a person taking the medicament,

b) receiving data suitable for a description of the actual effect of the medicament,

c) storing the data suitable for the description of the actual effect of the medicament in the electronic monitoring file, and

d) comparing the data suitable for the description of the actual effect of the medicament with data suitable for a description of an expected effect of the medicament.

Thus, according to the invention, the electronic monitoring file which is allocated to the person taking the medicament, and in which the data suitable for the description of the actual effect of the medicament are stored, is initially set up by, for example, the physician who is treating the person. However, the setting-up of the electronic monitoring file and thus the remote monitoring of the effect of the medicament can also be offered as a service to the person by, for example, the manufacturer of the medicament, the person's medical provider or health insurance provider, or by an independent service provider. This offer can be advertised by printing the necessary information, for example the address of the institution providing the service, on a pack insert included with the medicament.

When setting up the monitoring file, not only is the identity of the person taking the medicament possibly rendered anonymous, but monitoring parameters for the remote monitoring are also established. These monitoring parameters correspond to the data describing the expected effect of the medicament, with which the data describing the actual effect of the medicament are compared according to the invention. Since the person generally takes the medicament so that his state of health improves or at least does not deteriorate, and the person therefore takes the medicament for a specific purpose, the desired effect, i.e. the expected effect, of the medicament is consequently also known. The data suitable for the description of the expected effect of the medicament can for example, as is provided for in one variant of the invention, be allocated to a diagnosis, on the basis of which the person is taking the medicament, to a dose of the medicament and/or to a side effect that is to be expected. However, as is provided for according to a further embodiment of the invention, an expected effect of the medicament can also be an expected medical state of the person taking the medicament. To ensure that efficient remote monitoring can be carried out, it is additionally possible, when setting up the monitoring file, to define times or time intervals at which the data suitable for the actual description of the effect of the medicament are to be received.

In the case of a person with more than one medical condition and/or taking several medicaments, an expert system can also be used which, from the diagnoses, medicaments and dosages of the individual medicaments, determines data suitable for the description of the expected effects of the medicaments.

During the period of the remote monitoring, the data suitable for the description of the actual effect of the medicament are received and stored in the electronic monitoring file. According to one variant of the invention, the actual effect of the medicament can be described, for example, by an actual medical state of the person taking the medicament. According to one embodiment of the invention, the description of the actual medical state can be determined, for example, on the basis of blood pressure, heart rate, nausea, bowel movement, blood values and/or the general well-being of the person taking the medicament.

According to one variant of the invention in particular, the actual medical state of the person taking the medicament is determined using at least one measurement appliance. For example, the institution setting up the electronic monitoring file can provide the person taking the medicament with the measurement appliance for the duration of the remote monitoring.

According to one variant of the invention, the actual medical state of the person taking the medicament is determined by said person himself, or by another person working in particular in the health care services.

According to one embodiment of the invention, the data suitable for the description of the actual effect of the medicament are advantageously transmitted via a data transmission device to a data processing unit holding the electronic monitoring file. A data transmission device is, for example, a telephone, a fax machine or the Internet. The person, or the other person working in particular in the health care services, can thus phone through the actual effect of the medicament, for example to the institution which set up the electronic monitoring file and is carrying out the remote monitoring. It is also conceivable for said institution to make available, on the data processing unit, a web page allocated to the person taking the medicament. The web page includes, for example, a questionnaire with which it is possible to determine the actual effect of the medicament. The person taking the medicament, or the other person working in particular in the health care services, can download this web page using a PC connected to the Internet, complete it and send the completed web page to the data processing unit. Straightforward and reliable transmission of the data describing the actual effect of the medicament is thus possible.

According to a further embodiment of the invention, an alarm signal is triggered if the actual effect of the medicament deviates from the expected effect by more than a predetermined extent. For example, if the desired effect of the medicament is not obtained, the alarm signal is reliably triggered. The alarm signal can be transmitted, for example, to the physician who is treating the person, to a hospital, or alternatively to relatives of the person taking the medicament. Thus, the person taking the medicament can obtain assistance particularly quickly if, after taking the medicament, the actual medical state of said person gives cause for concern.

According to one embodiment of the invention, the medicament is taken during a study of the efficacy of the medicament, in particular during phases III and IV. Studies of the efficacy of medicaments are divided into four phases. Phase I basically involves animal tests and individual trials on humans. Phase II involves small groups of humans taking the medicament under close clinical supervision during the study of the tolerance of the medicament. Phase III is by contrast a double-blind study which is required for approval, for example by the FDA (US Food and Drug Administration) or the Bundesamt fur Arzneimittel (German Federal Drugs Office). Phase IV involves a study intended to ascertain whether a medicament already approved for a specified medical indication may also be approved for other medical indications.

The study of the efficacy of a medicament can be carried out in particular in an advantageous manner if, according to a further embodiment of the invention, a web page is provided by means of which in particular persons taking the medicament can obtain information concerning any side effects arising during the study of the tolerance of the medicament.

According to a further variant of the invention, a web page is also provided on which persons taking the medicament can report any side effects caused by taking the medicament during the study of the tolerance of the medicament.

The second object of the invention is achieved by a system for remote monitoring of the effect of a medicament which a person is taking, said system having a data bank for storing an electronic monitoring file allocated to the person and to the medicament and for storing data suitable for a description of an expected effect of the medicament, means for receiving data suitable for the description of the actual effect of the medicament, and means for comparing the data suitable for the description of the actual effect of the medicament with the data suitable for the description of the expected effect of the medicament. Advantageous embodiments of the system according to the invention are set out in the subclaims.

The system according to the invention is therefore so configured that it can be used to carry out the method according to the invention.

The object of the invention is also achieved by a computer program which implements the method according to the invention, and a computer program which implements the system according to the invention. The object of the invention is likewise achieved by a data processing unit on which one of the computer programs according to the invention is implemented.

A basic concept of the invention is to couple the prescribing and taking of a medicament with remote monitoring of the effect of the medicament. The remote monitoring can be offered as a service. The data suitable for the description of the expected effect of the medicament can be derived from the diagnosis or diagnoses regarding the person taking the medicament, from the indication, from anticipated side effects of taking the medicament, and from a contraindication of the medicament.

If, for example, the medicament is a beta blocker which the person is taking to lower his blood pressure because he suffers from high blood pressure, the data suitable for the description of the actual effect of the medicament, i.e. of the beta blocker, include a measured blood pressure and a measured heart rate of the person because, when taking a beta blocker, bradycardia (slow heartbeat) may occur as a side effect. A further side effect which may occur when taking a beta blocker is impaired orthostatic control (dizziness, particularly when standing up). Consequently, dizziness can in particular be used as an indicator of the general well-being of the person.

An advantage of the invention is that the person taking the medicament can be monitored reliably and can thus be better medicated. The effect of the medicament and any side effects or complications can therefore be reliably detected and recorded, and, as a result, suitable countermeasures can be taken promptly.

The remote monitoring service can be offered by various providers working in isolation or cooperating with one another.

### BRIEF DESCRIPTION OF THE DRAWINGS

Illustrative embodiments are shown by way of example in the diagrammatic drawings, in which:

FIG. 1 shows a scenario illustrating the method according to the invention and the system according to the invention,

FIG. 2 shows a flowchart illustrating the method according to the invention,

FIG. 3 shows a web page, and

FIG. 4 shows a diagram illustrating the setting-up of an electronic monitoring file.

### DETAILED DESCRIPTION OF THE INVENTION

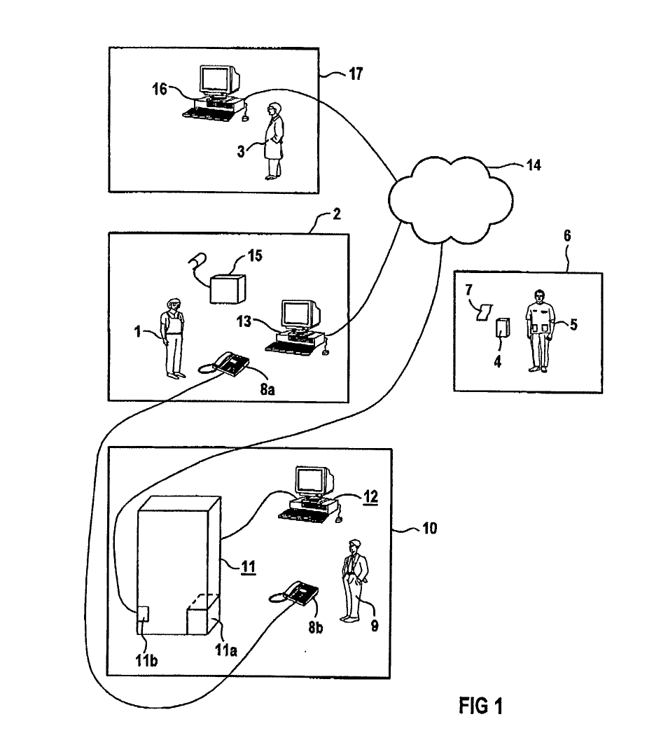

FIG. 1 shows a patient 1 who, in the case of the present illustrative embodiment, lives at his home 2 and suffers from high blood pressure. For this reason, he consulted a physician 3 in the latter's surgery 17. The physician 3 examined the patient 1 and prescribed him an antihypertensive medicament (beta blocker 4) which the patient 1 collected from a pharmacist 5 at the latter's pharmacy 6.

In the case of the present illustrative embodiment, the beta blocker 4 comes with a pack insert 7 which mentions an offer by the manufacturer of the beta blocker 4 to remotely monitor the effect of the beta blocker 4 while it is being taken. To accept the offer of remote monitoring, the patient 1 uses a telephone 8a located at his home 2 to dial a telephone number indicated on the pack insert 7, whereupon an employee 9 of the manufacturer of the beta blocker 4 answers from a telephone 8b located at the business premises 10 of the manufacturer.

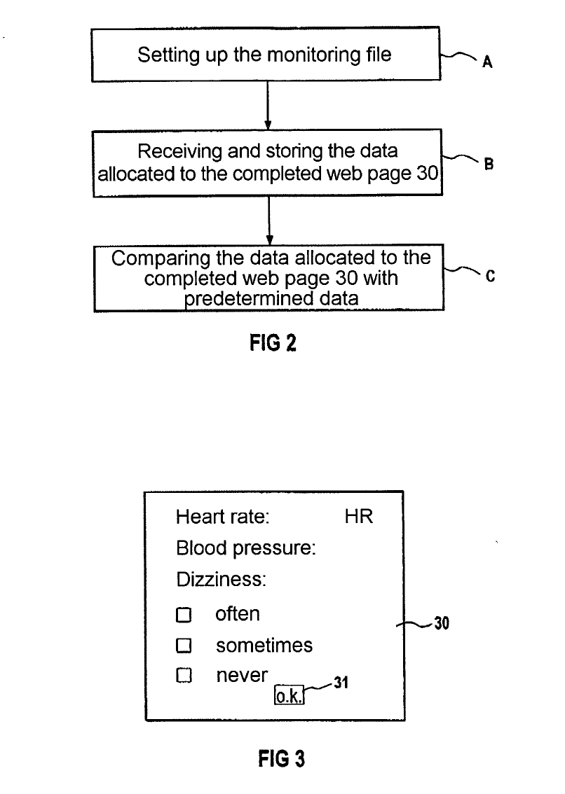

In the case of the present illustrative embodiment, after the employee has been told by the patient 1 that said patient 1 suffers from high blood pressure, has been prescribed the beta blocker 4 by the physician 3 and would like to accept the offer of remote monitoring, the employee 9 uses a PC 12 located at the business premises 10 to set up a monitoring file allocated to the patient 1. In the case of the present illustrative embodiment, the monitoring file is stored in a data bank 11a of a data processing unit 11 connected to the PC 12 (step A of the flowchart represented in FIG. 2).

To ensure that the employee 9 can set up a suitable remote monitoring program for the patient 1, i.e. can establish suitable monitoring parameters, the data bank 11a in the case of the present illustrative embodiment contains information on medicaments marketed by the manufacturer. In the case of the present illustrative embodiment, this information includes, among other things, details on reasons for taking the medicaments and on the desired effect and possible side effects of the medicaments sold by the manufacturer.

In the case of the present illustrative embodiment, the employee 9 determines, on the basis of the information stored in the data bank lla, that bradycardia (slow heartbeat) and impaired orthostatic control (dizziness, especially when standing up) may occur as undesirable side effects of taking the beta blocker 4. The desired effect of the beta blocker 4 is normalization of blood pressure. Consequently, the employee 9 informs the patient 1 that the patient 1 should measure his blood pressure and heart rate once daily and should send details of the measured blood pressure and the measured heart rate each day to the data processing unit 11.

So that the patient 1 is able to measure his blood pressure, the employee 9 provides him with a blood measurement appliance 15 for the duration of the remote monitoring. To ensure that the measured blood pressure and the measured heart rate can be transmitted reliably and in a way which is convenient for the patient 1, the employee 9, in the case of the present illustrative embodiment, creates a web page 30 (shown in FIG. 3) for the patient 1, which web page 30 is stored in the data processing unit 11. In the case of the present illustrative embodiment, the patient 1 can download the web page 30 in a generally known manner from the data processing unit 11 using a PC 13 which is connected to the Internet 14 and is located at the home 2 of the patient 1. The data processing unit 11 is likewise connected to the Internet 14 via a suitable Internet connection 11b.

In the case of the present illustrative embodiment, the patient 1 thus measures his blood pressure and his heart rate each day and enters the measured values in the web page 30 which he downloads each day from the data processing unit 11 using his PC 13. By means of the web page 30, the patient 1 also indicates whether he experiences dizziness often, sometimes or never. The completed web page 30 thus contains details or data suitable for the description of the effect of the beta blocker 4.

After the patient 1 has completed the web page 30, he sends the completed web page 30 to the data processing unit 11 by clicking on the field 31 of the web page 30 in a generally known manner using a computer mouse of the PC 13.

The data processing unit 11 also runs a suitable computer program which ensures that data intended for remote monitoring of customers of the manufacturer are entered at the correct times. In the case of the present illustrative embodiment, the computer program running on the data processing unit 11 monitors whether the patient 1 sends a completed web page 30 each day to the data processing unit 11. If the patient 1 omits to send a completed web page 30, the computer program automatically generates an e-mail which requests that the completed web page 30 be transmitted and which is automatically sent to the PC 13 of the patient 1.

After the completed web page 30 has been received by the data processing unit 11 and been stored in the data bank 11a, the computer program running on the data processing unit 11 checks whether the beta blocker 4 is acting in the desired manner, in other words whether the patient's blood pressure has normalized, and whether the undesirable side effects occur, in other words whether the patient's heart rate is too slow or whether the patient 1 often feels dizzy. The computer program executes this check by comparing the value of the measured blood pressure with a value which corresponds to a normal blood pressure, and comparing the value of the measured heart rate with a value which corresponds to a slow but still normal heart rate, and checks whether the patient 1 indicated that he often feels dizzy (steps B and C of the flowchart shown in FIG. 2).

If, in the case of the present illustrative embodiment, the check reveals that the desired effect of the beta blocker 4 does not occur or that a side effect occurs, the computer program running on the data processing unit 11 automatically generates a report. In the case of the present illustrative embodiment, this report contains details of whether a side effect occurred, and if so which one, and that the desired effect of the beta blocker 4 did not occur. In the case of the present illustrative embodiment, this report is sent in the form of an e-mail from the data processing unit 11 to a PC 16 which is likewise connected to the Internet 14 and is located at the surgery 17 of the physician 3 who is treating the patient 1. The physician 3 is thus reliably informed of whether the beta blocker 4 is having the desired effect or whether undesirable side effects are occurring and he is therefore able, if need be, to react appropriately.

After the remote monitoring of the patient 1 has ended, a further computer program stored in the data processing unit 11 automatically generates an account allocated to the remote monitoring of the patient 1, which account is then sent to the patient's health insurance provider.

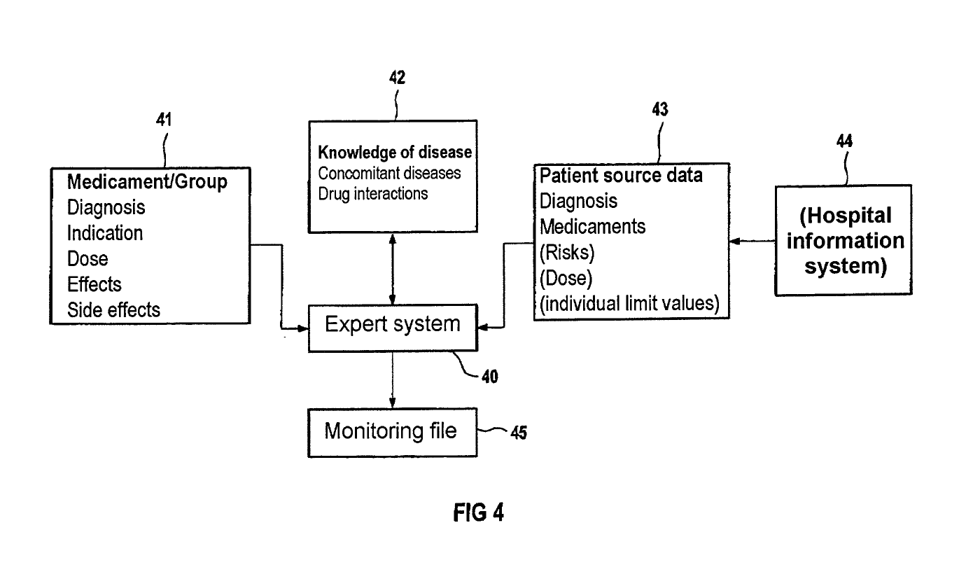

In the illustrative embodiment described above, the employee 9 determines the monitoring parameters suitable for remote monitoring of the patient 1 on the basis of the information stored in the data bank lla of the data processing unit 11. However, the monitoring parameters can also be determined by means of an expert system 40, as is shown diagrammatically and by way of example in FIG. 4.

So that the expert system 40 shown in FIG. 4 is able to determine suitable monitoring parameters, it is able, in the case of the present illustrative embodiment, to access data stored in lists 41, 42 and 43. The lists 41, 42 and 43 are, for example, stored in a suitable data bank.

In the case of the present illustrative embodiment, the list 41 contains details of the prescribed medicament or group of medicaments. These details include information concerning a diagnosis on the basis of which the medicament was prescribed, an indication, a dose, desired effects, undesirable side effects, etc. The necessary measurement parameters and the frequencies of the measurements can be determined from this information.

In the case of the present illustrative embodiment, the list 42 contains details of the knowledge of a disease, for example including information on drug interactions or concomitant diseases.

In the case of the present illustrative embodiment, the list 43 contains patient source data which are allocated to the patient 1 to be monitored and contain information concerning a diagnosis of the patient 1, medicaments being taken by the patient 1, possible risks, dose, or individual limit values. As is shown in FIG. 4, the patient source data can also be made available from a hospital information system 44.

On the basis of the data stored in the lists 41, 42 and 43, the expert system 40 can generate the data necessary for monitoring the patient 1 and can thus set up a suitable monitoring file 45.

If several medicaments are prescribed to patients with more than one disease, it may be, for example, that the same monitoring parameter is determined for two different medicaments. Different measurement intervals may be necessary in these cases, depending for example on the half-life of the medicaments. The expert system 40 recognizes such situations and, if necessary, can calculate suitable measurement periods.

The method according to the invention and the system according to the invention can be used for remote monitoring of various medicaments. The beta blocker 4 is given only as an example. In particular, the method according to the invention or the system according to the invention can be used for medicaments for treating chronic diseases (hypertension, asthma, COLD, diabetes, cardiac insufficiency, epilepsy, etc.), medicaments for treating acute conditions (for example pain or diarrhea), and for determining side effects of medicaments in the context of cancer therapy or in the context of treatment of pain.

For studies, in particular for studies of the efficacy of a medicament, study protocols can be established during the remote monitoring. On the basis of the study protocols, it is possible to determine which data are to be recorded by the person being monitored and at which times. During the study of the tolerance of a medicament, it is then possible in particular to store, in the data processing unit 11, a web page by means of which persons participating in the drug tolerance study can obtain information on any side effects of medicaments in the context of the drug tolerance study. A further web page can also be provided in the data processing unit 11, however, on which web page the persons participating in the drug tolerance study can report any side effects.

The illustrative embodiments are otherwise to be understood only as examples.

1. A method for remote monitoring of the effect of a medicament, said method comprising the following method steps:
a) setting up an electronic monitoring file which is allocated to a person taking the medicament,
b) receiving data suitable for a description of the actual effect of the medicament,
c) storing the data suitable for the description of the actual effect of the medicament in the electronic monitoring file, and
d) comparing the data suitable for the description of the actual effect of the medicament with data suitable for a description of an expected effect of the medicament.

2. The method as claimed in claim 1, wherein the data suitable for the description of the expected effect of the medicament are allocated to a diagnosis, on the basis of which the person is taking the medicament, to a dose of the medicament and/or to a side effect that is to be expected.

3. The method as claimed in claim 1, wherein the expected effect of the medicament is an expected medical state of the person taking the medicament.

4. The method as claimed in claim 1, wherein the description of the actual effect of the medicament is a description of an actual medical state of the person taking the medicament.

5. The method as claimed in claim 4, wherein the actual medical state of the person is determined on the basis of blood pressure, heart rate, nausea, bowel movement, blood values and/or the general well-being of the person taking the medicament.

6. The method as claimed in claim 4, wherein the actual medical state of the person is determined using at least one measurement appliance.

7. The method as claimed in claim 4, wherein the actual medical state is determined by the person taking the medicament, or by another person working in particular in the health care services.

8. The method as claimed in claim 1, wherein the data suitable for the description of the actual effect of the medicament are transmitted via a data transmission device to a data processing unit holding the electronic monitoring file.

9. The method as claimed in claim 1, wherein an alarm signal is triggered if the effect of the medicament deviates from the expected effect by more than a predetermined extent.

10. The method as claimed in claim 1, wherein the medicament is taken during a study of the efficacy of the medicament, in particular during phases III and IV.

11. The method as claimed in claim 10, wherein a web page is provided by means of which in particular persons taking the medicament can obtain information concerning any side effects arising during the study of the tolerance of the medicament.

12. The method as claimed in claim 10, wherein a web page is provided on which persons taking the medicament can report any side effects caused by taking the medicament during the study of the tolerance of the medicament.

13. A system for remote monitoring of the effect of a medicament which a person is taking, said system comprising
a data bank for storing an electronic monitoring file allocated to the person and to the medicament and for storing data suitable for a description of an expected effect of the medicament,
means for receiving data suitable for the description of the actual effect of the medicament, and
means for comparing the data suitable for the description of the actual effect of the medicament with the data suitable for the description of the expected effect of the medicament..

14. The system as claimed in claim 13, wherein the data suitable for the description of the expected effect of the medicament are allocated to a diagnosis, on the basis of which the person is taking the medicament, to a dose of the medicament and/or to a side effect that is to be expected.

15. The system as claimed in claim 13, wherein the expected effect of the medicament is an expected medical state of the person taking the medicament.

16. The system as claimed in claim 13, wherein the description of the actual effect of the medicament is a description of an actual medical state of the person taking the medicament.

17. The system as claimed in claim 16, wherein the actual medical state is described on the basis of blood pressure, heart rate, nausea, bowel movement, blood values and/or the general well-being of the person taking the medicament.

18. The system as claimed in claim 16, wherein the actual medical state is determined using at least one measurement appliance.

19. The system as claimed in claim 13, wherein the actual effect of the medicament is determined by the person taking the medicament, or by another person working in particular in the health care services.

20. The system as claimed in claim 13, wherein the means for receiving data suitable for the description of the actual effect of the medicament are configured in such a way that the data suitable for the description of the actual effect of the medicament can be transmitted via a data transmission device.

21. The system as claimed in claim 13, further comprising means for triggering an alarm signal if the effect of the medicament deviates from the expected effect by more than a predetermined extent.

22. The system as claimed in claim 13, wherein said system is suitable for monitoring the effect of the medicament during a study of the efficacy of the medicament, during phases III and IV.

23. A computer program, which implements the method as claimed in claim 1.

24. A computer program, which implements the system as claimed in claim 13.

25. A data processing unit, on which a computer program as claimed in claim 23 is stored.

26. A data processing unit, on which a computer program as claimed in claim 24 is stored.
